(19) United States
(12) Patent Application Publication
(10) Pub. No.: US 2020/0132546 A1
(43) Pub. Date: Apr. 30, 2020

(54) METHOD FOR DETECTING SULFUR HEXAFLUORIDE

(71) Applicant: Quantum Spatial, Inc., St. Petersburg, FL (US)

(72) Inventor: Scott Nowicki, Portland, OR (US)

(21) Appl. No.: 16/669,388

(22) Filed: Oct. 30, 2019

Related U.S. Application Data
(60) Provisional application No. 62/752,975, filed on Oct. 30, 2018.

(51) Int. Cl. G01J 3/28; G01J 3/42; C01B 17/45

(52) U.S. Cl. CPC G01J 3/2823; G01J 3/42; G01N 33/0052; G01J 2003/2826; C01B 17/453

(57) ABSTRACT

Various embodiments are directed to a method of utilizing an imaging system for detecting a greenhouse gas such as sulfur hexafluoride. The method may include (1) generating, by a first thermal camera coupled to a robotic platform, a static image of a scene utilizing a first spectral filter for passing wavelengths within an SF6 absorption range, (2) generating, by a second thermal camera coupled to the robotic platform, an additional static image of the scene utilizing a second spectral filter for passing wavelengths outside of the SF6 absorption range, and (3) detecting the presence of SF6 in the scene based, at least in part, on the static image, the additional static image, and a difference between the static image and the additional static image.

### CROSS-REFERENCE TO RELATED APPLICATION

This application claims the benefit of U.S. Provisional Application No. 62/752,975, filed Oct. 30, 2018, the disclosure of which is incorporated, in its entirety, by this reference.

### TECHNICAL FIELD

Embodiments of the disclosure relate generally to thermal or greenhouse gas detection, and more specifically, to the use of an imaging system to detect the presence of sulfur hexafluoride gas.

### BACKGROUND

Sulfur hexafluoride (SF6) is a thermally active greenhouse gas that is often used as an insulating medium in high capacity transmission circuits in which leaks may often occur. SF6 behaves similarly to other greenhouse gases, where the lack of short-wavelength spectral absorptions results in an optically transparent plume at any gas concentration, and a major vibrational absorption feature causes the gas to be easily identified in the long-wavelength (thermal infrared) portion of the spectrum even at low concentrations.

Conventional techniques for performing SF6 detection often includes an inspector utilizing a hand-held video camera including a cooled thermal detector with a tuned spectral filter in the optical path configured to display imagery (from a single perspective). While these techniques may enable an inspector to visualize the location of a gas leak or plume, they are not capable of diagnostically determining the presence or concentration of the gas. Thus, the use of the aforementioned hand-held camera types is often not suitable for machine-vision or automated detection, as an inspector needs to interpret and visually verify the presence of the anomalies (e.g., a leak) in a complex thermal scene. It is with respect to these considerations and others that the various embodiments of the present invention have been made.

### SUMMARY

As will be described in greater detail below, the instant disclosure generally relates to a method for detecting sulfur hexafluoride (SF6) utilizing an imaging system. In one example, the method may include (1) generating, by a first thermal camera coupled to a robotic platform, a static image of a scene utilizing a first spectral filter for passing wavelengths within an SF6 absorption range, (2) generating, by a second thermal camera coupled to the robotic platform, an additional static image of the scene utilizing a second spectral filter for passing wavelengths outside of the SF6 absorption range, and (3) detecting the presence of SF6 in the scene based, at least in part, on the static image, the additional static image, and a difference between the static image and the additional static image.

In some examples, the static image of the scene utilizing the first spectral filter may be generated by utilizing an uncooled thermal detector with the first spectral filter in an optical path for passing the wavelengths within the SF6 absorption range. In some examples, the static image of the scene utilizing the second spectral filter may be generated by utilizing another uncooled thermal detector with the second spectral filter in another optical path for passing the wavelengths outside of the SF6 absorption range, where the second thermal camera is aligned with the first thermal camera, and where the first and second thermal cameras are operative to co-collect long exposure images. In some examples, the long exposure images may include images captured over an extended time period. In one example, the extended time period may be about 500 milliseconds.

In some examples, the first spectral filter may include a band-pass filter that passes the wavelengths within the SF6 absorption range and the second spectral filter may include a band-pass filter that passes the wavelengths outside of the SF6 absorption range. In some embodiments, the difference between the static image and the additional static image provide one or more independent observations for autonomously detecting a presence of the SF6 in the scene. In some examples, the presence of the SF6 in the scene may correspond to a detection of a leak in one or more high capacity transmission circuits utilized in an electrical power station.

Features from any of the above-mentioned embodiments may be used in combination with one another in accordance with the general principles described herein. These and other embodiments, features, and advantages will be more fully understood upon reading the following detailed description in conjunction with the accompanying drawings and claims.

### BRIEF DESCRIPTION OF THE FIGURES

FIG. 1 illustrates a block diagram of an example imaging system that may be utilized in accordance with various embodiments.

FIG. 2 illustrates a block diagram of spectral filter components in the example imaging system of FIG. 1, in accordance with an example embodiment.

FIG. 3 illustrates a block diagram of another example imaging system that may be utilized in accordance with various embodiments.

FIG. 4 is a flow diagram illustrating a method of detecting SF6 utilizing the example imaging system of FIG. 1, according to an example embodiment.

### DETAILED DESCRIPTION

The present disclosure describes a method of detecting sulfur hexafluoride (SF6) utilizing an imaging system. The imaging system may utilize co-aligned thermal cameras to enable automated machine-vision detection of an SF6 plume or leak. The embodiments of the disclosure described herein provide several advantages over conventional techniques utilizing handheld video cameras consisting of a single cooled thermal detector with a single spectral filter utilized by a human inspector. These advantages include utilizing a multispectral approach to accurately and automatically detect SF6 plumes or leaks utilizing an imaging system, including cameras with uncooled thermal detectors and two different spectral filters, mounted to a robotic platform. This type of detection would not be able to be performed using the aforementioned conventional techniques as the handheld camera types utilized by human inspectors only provide a single-image perspective and therefore are unsuitable for machine vision or automated detection. Moreover, the components of the aforementioned imaging system may be implemented at a significantly lower cost than conventional systems and may be adapted for use on a variety of robotic platforms.

Embodiments of the disclosure now will be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

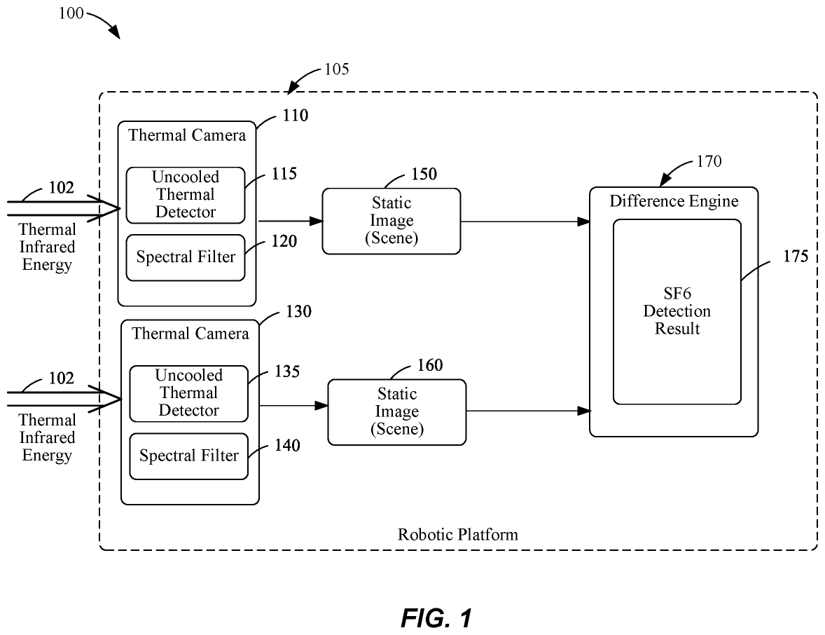

FIG. 1 illustrates a block diagram of an example imaging system 100. In some examples, imaging system 100 may include two co-aligned thermal cameras 110 and 130 mounted to a robotic platform 105. In some embodiments, robotic platform 105 may be utilized for performing various tasks, such as detecting thermal infrared energy 102 (which may include SF6) during a facility inspection. For example, the facility may include high capacity transmission circuits for which SF6 is used as an insulating medium.

In some embodiments, thermal camera 110 may include an uncooled thermal detector 115 and a spectral filter 120. Thermal camera 110 may utilize uncooled thermal detector 115 to generate static imagery of a scene, such as static image 150, thereby eliminating the need for a cooled detector. In one example, static image 150 may be a still image taken of thermal infrared energy 102 utilizing uncooled thermal detector 115. Spectral filter 120 may include a band-pass filter within an SF6 absorption range. In some examples, spectral filter 120 may be in an optical path between thermal camera 115 and thermal infrared energy 102 in a scene such that static image 150 contains a filtered image of thermal infrared energy 102 within the SF6 absorption range.

In some embodiments, thermal camera 130 may include an uncooled thermal detector 135 and a spectral filter 140. Thermal camera 130 may utilize uncooled thermal detector 135 to generate static imagery of a scene, such as static image 160, thereby eliminating the need for a cooled detector. In one example, static image 160 may be a still image taken of thermal infrared energy 102 utilizing uncooled thermal detector 135. In some examples, spectral filter 140 may include a band-pass filter outside of an SF6 absorption range. In some examples, spectral filter 140 may be in an optical path between thermal camera 130 and thermal infrared energy 102 in a scene such that static image 160 contains a filtered image of thermal infrared energy 102 outside of the SF6 absorption range. In some examples, thermal cameras 115 and 130 may be configured to co-collect static images 150 and 160 as long exposure images (e.g., approximately 500 milliseconds).

In some embodiments, imaging system 100 may also include a difference engine 170. In one example, difference engine 170 may be configured to automatically detect the presence of a greenhouse gas, such as SF6, in a scene based on static images 150 and 160 and output an SF6 detection result 175. For example, difference engine 170 may be configured to utilize machine vision techniques to automatically analyze static images 150 and 160 to determine differences based on static images 150 and 160 containing filtered images of thermal infrared energy 102 within the SF6 absorption range and outside of the SF6 absorption range, respectively. In one embodiment, difference engine 170 may be implemented as hardware and/or software on a computing device containing a processor and a memory configured to analyze static images and extract information based on the analysis.

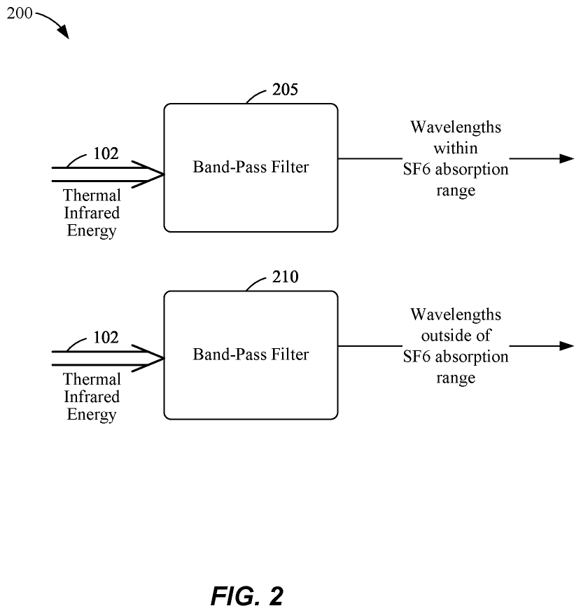

FIG. 2 illustrates a block diagram of spectral filter components 200 in the example imaging system of FIG. 1, in accordance with an example embodiment. As shown in FIG. 2, spectral filter components 200 may include a band-pass filter 205 and a band-pass filter 210. In some embodiments, band-pass filter 205 may represent or be incorporated into spectral filter 120 of FIG. 1. Similarly, band-pass filter 210 may represent or be incorporated into spectral filter 140 of FIG. 1. In some examples, band-pass filter 205 may be configured to filter thermal infrared energy 102 such that only wavelengths within the SF6 absorption range are passed through. Similarly, band-pass filter 210 may be configured to filter thermal infrared energy 102 such that only wavelengths outside of the SF6 absorption range are passed through.

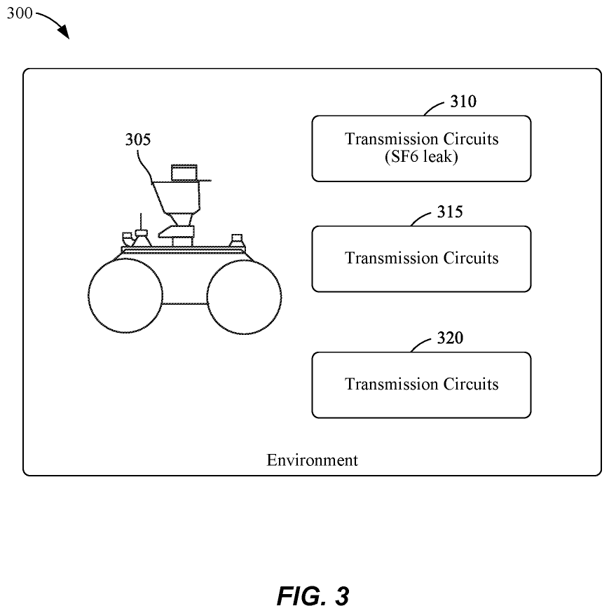

FIG. 3 illustrates a block diagram of an example imaging system 300 that may be utilized in accordance with various embodiments. As shown in FIG. 3, imaging system 300 may include a robotic platform 305 (which may correspond to and include all of the components described with respect to the robotic platform 105 of FIG. 1) and a group of transmission circuits 310, 315, and 310. In some examples, robotic platform 305 may be performing a facility inspection in an environment including a facility substation (such as an electrical power station) containing high capacity transmission circuits (e.g., transmission circuits 310, 315, and 320). In one embodiment, robotic platform 305 may utilize the imaging system described in FIG. 1 to detect SF6 leaks in transmission circuits 310, 315, and 310. For example, robotic platform 305 may travel within the environment (e.g., the facility substation) and utilize thermal cameras 110 and 130 of FIG. 1 to generate static images of any thermal infrared energy that may be present for input into difference engine 170 (of FIG. 1) to detect the presence of SF6 leaking from transmission circuits 310.

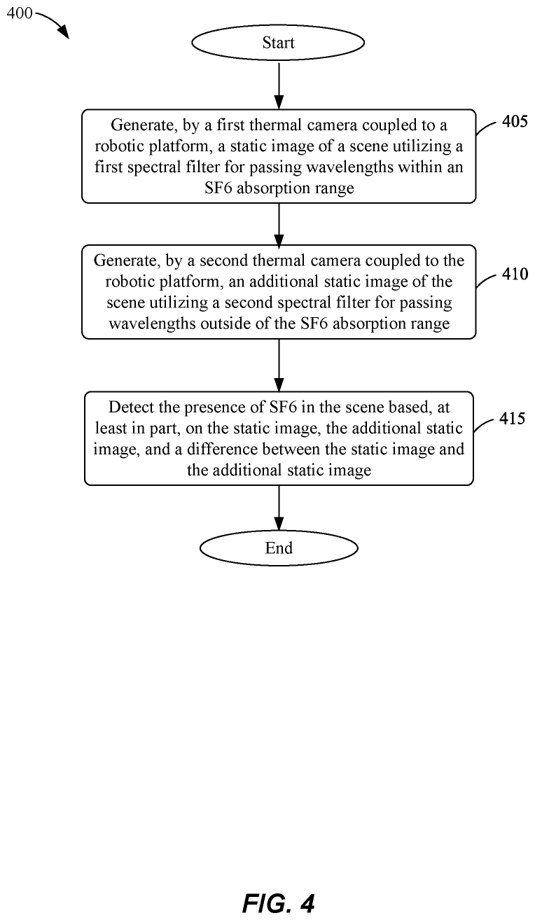

FIG. 4 is a flow diagram of an example method 400 for detecting SF6 utilizing an imaging system. As illustrated in FIG. 4, at step 402 one or more of the systems described herein (e.g., imaging system 100 of FIG. 1) may generate, by a first thermal camera coupled to a robotic platform, a static image of a scene utilizing a first spectral filter for passing wavelengths within an SF6 absorption range. For example, thermal camera 110 of FIG. 1 (which is coupled to robotic platform 105) may generate static image 150 utilizing spectral filter 120. In particular, static image 150 may be a still image taken of thermal infrared energy 102 utilizing uncooled thermal detector 115 in conjunction with spectral filter 120. Spectral filter 120 may include a band-pass filter within an SF6 absorption range. In some examples, spectral filter 120 may be in an optical path between thermal camera 115 and thermal infrared energy 102 in a scene such that static image 150 contains a filtered image of thermal infrared energy 102 within the SF6 absorption range.

At step 404, one or more of the systems described herein may generate, by a second thermal camera coupled to the robotic platform, an additional static image of the scene utilizing a second spectral filter for passing wavelengths outside of the SF6 absorption range. For example, thermal camera 130 of FIG. 1 (which is coupled to robotic platform 105) may generate static image 160 utilizing spectral filter 140. In particular, static image 160 may be a still image taken of thermal infrared energy 102 utilizing uncooled thermal detector 135 in conjunction with spectral filter 140. Spectral filter 140 may include a band-pass filter outside of the SF6 absorption range. In some examples, spectral filter 140 may be in an optical path between thermal camera 130 and thermal infrared energy 102 in a scene such that static image 160 contains a filtered image of thermal infrared energy 102 outside of the SF6 absorption range.

At step 406, one or more of the systems described herein may detect the presence of SF6 in the scene based, at least in part, on the static image, the additional static image, and a difference between the static image and the additional static image. For example, difference engine 170 of FIG. 1 may be utilized to difference engine 170 may be configured to automatically detect the presence of SF6 in the scene based on static images 150 and 160 and output an SF6 detection result 175. In some examples, difference engine 170 may be configured to utilize machine vision techniques to automatically analyze static images 150 and 160 to determine differences based on static images 150 and 160 containing filtered images of thermal infrared energy 102 within the SF6 absorption range and outside of the SF6 absorption range, respectively.

In some embodiments, the SF6 imaging system described above for automated and accurate leak detection may be constructed from a range of various components including, without limitation, uncooled thermal detectors, optics (e.g., optical lenses), and spectral filters. In some examples, the components utilized in the SF6 imaging system may be selected for optimal signal to noise ratio, resolution and field of view, and temperature range.

In some examples, the SF6 imaging system described herein may include a robotic platform coupled to the thermal cameras to facilitate operations associated with performing automated or semi-automated inspections of facility substations. In one embodiment, the SF6 imaging system may be utilized for leak detection associated with high capacity transmission circuits utilized in an electrical power station.

The term “robotic platform” as used herein, generally refers to any form of machine, programmable by a computer, capable of autonomously or semi-autonomously carrying out a complex series of actions or tasks such as facility inspections. Examples of robotic platforms may include, without limitation, robots, aquatic mobility systems (e.g., autonomous surface vehicles), surface-based mobility systems (e.g., unmanned ground vehicles (UGVs) including autonomous cars, etc.), and/or other programmable mobile machines that are capable of directional movement on the ground or on water.

The robotic platforms disclosed herein may be utilized in a variety of environments and conditions, including, for example facility substations (e.g., electrical and/or water utility substations), data centers, industrial environments (e.g., factories, plants, etc.), warehouses (e.g., storage warehouses, shipping warehouses, etc.), construction sites, buildings, outdoor spaces, and/or any other suitable environment or location, without limitation.

The preceding description has been provided to enable others skilled in the art to best utilize various aspects of the exemplary embodiments disclosed herein. This exemplary description is not intended to be exhaustive or to be limited to any precise form disclosed. Many modifications and variations are possible without departing from the spirit and scope of the instant disclosure. The embodiments disclosed herein should be considered in all respects illustrative and not restrictive. Reference should be made to the appended claims and their equivalents in determining the scope of the instant disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and have the same meaning as the word “comprising.”

What is claimed is:

1. A method for utilizing an imaging system to detect sulfur hexafluoride (SF6), comprising:
generating, by a first thermal camera coupled to a robotic platform, a static image of a scene utilizing a first spectral filter for passing wavelengths within an SF6 absorption range;
generating, by a second thermal camera coupled to the robotic platform, an additional static image of the scene utilizing a second spectral filter for passing wavelengths outside of the SF6 absorption range; and
detecting a presence of SF6 in the scene based, at least in part, on the static image, the additional static image, and a difference between the static image and the additional static image.

2. The method of claim 1, wherein generating the static image of the scene utilizing the first spectral filter comprises utilizing an uncooled thermal detector with the first spectral filter in an optical path for passing the wavelengths within the SF6 absorption range.

3. The method of claim 1, wherein generating the additional static image of the scene utilizing another uncooled thermal detector with the second spectral filter in another optical path for passing the wavelengths outside of the SF6 absorption range, wherein the second thermal camera is aligned with the first thermal camera, and wherein the first and second thermal cameras are operative to co-collect long exposure images.

4. The method of claim 3, wherein the long exposure images comprise images captured over an extended time period.

5. The method of claim 4, wherein the extended time period comprises about 500 milliseconds.

6. The method of claim 1, wherein the first spectral filter comprises a band-pass filter that passes the wavelengths within the SF6 absorption range.

7. The method of claim 1, wherein the second spectral filter comprises a band-pass filter that passes the wavelengths outside of the SF6 absorption range.

8. The method of claim 1, wherein the difference between the static image and the additional static image provide one or more independent observations for autonomously detecting a presence of the SF6 in the scene.

9. The method of claim 8, wherein the presence of the SF6 in the scene corresponds to a detection of a leak in one or more high capacity transmission circuits utilized in an electrical power station.

10. A method for utilizing an imaging system to detect a greenhouse gas, comprising:
generating, by a first thermal camera coupled to a robotic platform, a static image of a scene utilizing a first spectral filter for passing wavelengths within an absorption range of the greenhouse gas;
generating, by a second thermal camera coupled to the robotic platform, an additional static image of the scene utilizing a second spectral filter for passing wavelengths outside of the absorption range of the greenhouse gas; and
detecting a presence of the greenhouse gas in the scene based, at least in part, on the static image, the additional static image, and a difference between the static image and the additional static image.

11. The method of claim 10, wherein generating the static image of the scene utilizing the first spectral filter comprises utilizing an uncooled thermal detector with the first spectral filter in an optical path for passing the wavelengths within the absorption range of the greenhouse gas.

12. The method of claim 10, wherein generating the additional static image of the scene utilizing another uncooled thermal detector with the second spectral filter in another optical path for passing the wavelengths outside of the absorption range of the greenhouse gas, wherein the second thermal camera is aligned with the first thermal camera, and wherein the first and second thermal cameras are operative to co-collect long exposure images.

13. The method of claim 12, wherein the long exposure images comprise images captured over an extended time period.

14. The method of claim 13, wherein the extended time period comprises about 500 milliseconds.

15. The method of claim 10, wherein the first spectral filter comprises a band-pass filter that passes the wavelengths within the absorption range of the greenhouse gas.

16. The method of claim 10, wherein the second spectral filter comprises a band-pass filter that passes the wavelengths outside of the absorption range of the greenhouse gas.

17. The method of claim 10, wherein the difference between the static image and the additional static image provide one or more independent observations for autonomously detecting a presence of the greenhouse gas in the scene.

18. The method of claim 17, wherein the presence of the greenhouse gas in the scene corresponds to a detection of a leak in one or more high capacity transmission circuits utilized in an electrical power station.

19. The method of claim 10, wherein the greenhouse gas comprises sulfur hexafluoride (SF6).

20. A method for utilizing an imaging system to detect sulfur hexafluoride (SF6), comprising:
generating, by a first thermal camera coupled to a robotic platform, a static image of a scene utilizing a first spectral filter for passing wavelengths within an SF6 absorption range, wherein generating the static image of the scene utilizing the first spectral filter comprises utilizing an uncooled thermal detector with the first spectral filter in an optical path for passing the wavelengths within the SF6 absorption range;
generating, by a second thermal camera coupled to the robotic platform, an additional static image of the scene utilizing a second spectral filter for passing wavelengths outside of the SF6 absorption range, wherein generating the additional static image of the scene utilizing another uncooled thermal detector with the second spectral filter in another optical path for passing the wavelengths outside of the SF6 absorption range, wherein the second thermal camera is aligned with the first thermal camera, and wherein the first and second thermal cameras are operative to co-collect long exposure images; and
detecting a presence of SF6 in the scene based, at least in part, on the static image, the additional static image, and a difference between the static image and the additional static image.
